(19) United States
(12) Patent Application Publication
(10) Pub. No.: US 2024/0398330 A1
(43) Pub. Date: Dec. 5, 2024

(54) WEARABLE DEVICE FOR REAL TIME MEASUREMENT OF SWALLOWING

(71) Applicants: A.Y.Y.T. - TECHNOLOGICAL APPLICATIONS AND DATA UPDATE LTD, HOLON (IL); Mor Research Applications Ltd., RAMAT GAN (IL)

(72) Inventors: Michal Balberg, Holon (IL); Jonathan Rubin, Holon (IL); Avihai Aharon, HOLON (IL); Heftsi Ragones, Holon (IL); Yael Avni, HOLON (IL); Daniil Umansky, HOLON (IL); Hagit Shoffel Havakuk, RAMAT GAN (IL); Saja Assi, RAMAT GAN (IL)

(73) Assignees: A.Y.Y.T. - TECHNOLOGICAL APPLICATIONS AND DATA UPDATE LTD, HOLON (IL); Mor Research Applications Ltd., RAMAT GAN (IL)

(21) Appl. No.: 18/695,851

(22) PCT Filed: Sep. 29, 2022
(86) PCT No.: PCT/IL2022/051038

§ 371 Date: Mar. 27, 2024

(30) Foreign Application Priority Data
Sep. 30, 2021 (IL) 286883

(51) Int. Cl. A61B 5/00; A61B 5/053; A61B 5/389; A61B 7/00

(52) U.S. Cl. CPC A61B 5/4205; A61B 5/053; A61B 5/389; A61B 5/6802; A61B 5/6822; A61B 7/008

(57) ABSTRACT

Disclosed herein is a multi-modal sensor system, including a wearable device configured to receive signals relating to a swallowing process of a subject, the wearable device including one or more surface Electromyograph sensors configured to receive signals relating to electrical potential in muscles of the throat, one or more bio-impedance sensors, one or more memories, one or more processors configured to operate one or more sensors of the wearable device, synchronize the signals to one or more predetermined events to generate a synchronization feature, receive the signals as a first diagnostic data set, analyze the first diagnostic data set, assess, based on the analysis, the swallowing process of the subject to yield an assessment output, and present the assessment output and determine a bio-impedance signal.

### FIELD OF THE INVENTION

The present disclosure generally relates to real time assessment of swallowing.

### BACKGROUND

Dysphagia can result from nerve or muscle problems. Conservative estimates suggest that dysphagia may be as high as 22% in adults over fifty. Dysphagia particularly impacts the elderly-50-70% of nursing home residents); patients with neurological diseases-35-50% in victims from stroke, traumatic brain injury, cranial nerve lesion; neurodegenerative diseases, such as Parkinson's disease, ALS, MS, Dementia and Alzheimer: 50-100%; and head and neck cancer-40-60% of cancer patients. If untreated, Dysphagia can cause bacterial aspiration, pneumonia, dehydration and malnutrition. Victims of this disorder can suffer pain, suffocation, recurrent pneumonia, gagging and other medical complications. In the United States, Dysphagia accounts for about 60,000 deaths annually.

Current diagnosis of the illness typically utilizes obtrusive endoscopy or radioactive fluoroscopy, and the treatment focuses on interventions through exercise and physiotherapy, most of which are performed in hospitals and clinics. The availability of these services is limited rural locations, and is mostly available in urban centers where the facilities are easily accessible, which requires subjects requiring treatment, which are usually elderly individuals, to travel to the dedicated facilities for diagnostic and treatment.

### SUMMARY

The following embodiments and aspects thereof are described and illustrated in conjunction with systems, tools and methods which are meant to be exemplary and illustrative, not limiting in scope.

There is provided, in accordance with an embodiment, a multi-modal sensor system, including a wearable device configured to receive signals relating to a swallowing process of a subject, the wearable device including one or more surface Electromyograph sensors configured to receive signals relating to electrical potential in muscles of the throat, one or more bio-impedance sensors, one or more memories, one or more processors configured to operate one or more sensors of the wearable device, synchronize the signals to one or more predetermined events to generate a synchronization feature, receive the signals as a first diagnostic data set, analyze the first diagnostic data set, assess, based on the analysis, the swallowing process of the subject to yield an assessment output, and present the assessment output and determine a bio-impedance signal.

In some embodiments, the one or more bio-impedance sensors is configured to receive signals relating to electric current flow in tissue of the throat in response to application of variable electric potential and the one or more processors are further configured to designate the signals received from the one or more bio-impedance sensors as bio-impedance signals.

In some embodiments, the one or more bio-impedance sensors is configured to receive signals related to biopotential in response to current flow in tissue of the throat and the one or more processors are further configured to designate the signals received from the one or more bio-impedance sensors as bio-impedance signals.

In some embodiments, the wearable device further including one or more mechanical sensors configured to receive signals relating to motion activity of the throat of the subject, and one or more microphones configured to collect audio signals relating to the throat of the subject.

In some embodiments, the one or more processors are further configured to analyze the bio-impedance signals to generate a time dependent tomographic map of the bio-impedance of a cross section of the throat.

In some embodiments, the assessment output includes a relation between the signals selected from the list which consists of surface Electromyography, bio-impedance, mechanical and audio signals.

In some embodiments, the assessment output includes a severity score.

In some embodiments, the one or more processors are further configured to wait a predetermined time period, receive collected signals for a second diagnostic data set, assess, by analyzing the second diagnostic data set and comparing with the first diagnostic data set, whether the swallowing process changed, and generate a second assessment output indicating progress of the swallowing process.

In some embodiments, the processor if further configured to initiate a user interface facilitate instructing the subject with a predetermined treatment, and updating instructions for the subject according to progress of the subject and the second assessment output.

In some embodiments, the processor is further configured to providing updated instructions according to the assessment output and input of a user.

In some embodiments, the assessment output includes a personalized treatment recommendation.

In some embodiments, the assessment output includes a condition prediction. In some embodiments, the system further includes a wireless communication unit configured to facilitate communication between the one or more processors and the one or more surface Electromyographs, one or more bio-impedance sensors, and one or more mechanical sensors and one or more audio sensors.

In some embodiments, the system, further including a display configured to show the display output.

In some embodiments, the one or more mechanical sensors is an accelerometer.

In some embodiments, the one or more mechanical sensors is a strain sensor.

In some embodiments, the wearable device further includes a double-sided disposable adhesive surface to facilitate fastening the wearable device to the neck throat of the subject.

In some embodiments, the one or more bio-impedance sensors includes a plurality of bio-impedance sensors positioned to surround the throat at least 300 degrees.

In some embodiments, the one or more surface Electromyographs and the one or more mechanical sensors are positioned adjacent to a Larynx of the subject.

In some embodiments, analysis of the signal includes measuring predetermined parameters of the signal.

In some embodiments, the analysis further includes determining a correlation between two or more signals of the signals collected.

In some embodiments, the predetermined event is a breathing cycle of the subject.

In some embodiments, the predetermined event is a characteristic of one or more signals relating to the swallowing process.

In some embodiments, the one or more processors are further configured to present a synchronization feature.

In some embodiments, the one or more processors are further configured to store collected signals.

There is further provided, in accordance with an embodiment a method including using one or more hardware processors for operating one or more sensors of a wearable device, synchronizing the signals to one or more predetermined events to generate a synchronization feature, receiving the signals as a first diagnostic data set, analyzing the first diagnostic data set, assessing, based on the analysis, the swallowing process of the subject to yield an assessment output, and presenting the assessment output.

In some embodiments, the method further including using the one or more processors for waiting a predetermined time period, receiving collected signals for a second diagnostic data set, assessing, by analyzing the second diagnostic data set and comparing with the first diagnostic data set, whether the swallowing process changed, and generating a second assessment output indicating progress of the subject.

In some embodiments, the method further including using the one or more processors for initiating a user interface to facilitate instructing the subject with a predetermined treatment, and updating instructions for the subject according to progress of the subject and the second assessment output.

In some embodiments, the signals are collected by a wearable device including one or more surface Electromyographs configured to receive signals relating to electrical potential in tissue of the throat, and one or more bio-impedance sensors configured to receive signals relating to electric current flow in response to application of variable electric potential in tissue of the throat.

In some embodiments, the wearable device further includes one or more mechanical sensors configured to receive signals relating to motion activity of the throat of the subject, and one or more microphones configured to collect audio signals relating to the throat of the subject.

In some embodiments, the assessment output includes a condition prediction.

In some embodiments, analyzing the signal includes measuring predetermined parameters of the signal

In some embodiments, the analyzing the signal further includes determining a correlation between at least two signals of the signals collected.

In some embodiments, the method further including presenting a synchronization feature.

### BRIEF DESCRIPTION OF THE DRAWINGS

Some non-limiting exemplary embodiments or features of the disclosed subject matter are illustrated in the following drawings.

Identical, duplicate, equivalent or similar structures, elements, or parts that appear in one or more drawings are generally labeled with the same reference numeral, optionally with an additional letter or letters to distinguish between similar entities or variants of entities, and may not be repeatedly labeled and/or described.

Dimensions of components and features shown in the figures are chosen for convenience or clarity of presentation and are not necessarily shown to scale or true perspective. For convenience or clarity, some elements or structures are not shown or shown only partially and/or with different perspective or from different point of views.

References to previously presented elements are implied without necessarily further citing the drawing or description in which they appear.

FIG. 1 schematically illustrates a system for real-time measuring of swallowing, according to certain exemplary embodiments;

FIGS. 2A-2B schematically illustrate a wearable device of the system of FIG. 1, according to certain exemplary embodiments;

FIG. 3 schematically illustrates a user interface presented on a display of the system of FIG. 1, according to certain exemplary embodiments

FIGS. 4A-4B outline operations of a method for assessing a dysphagia condition of a subject, according to certain exemplary embodiments;

FIG. 5 shows three graphs of EMG signals collected for different deglutition activities, according to certain exemplary embodiments;

FIG. 6A-6C shows three samples points on a bioimpedance tomography map, according to certain exemplary embodiments;

FIG. 7 shows a graph showing regions of interest as a function of time, according to certain exemplary embodiments;

FIG. 8 shows a graph of a surface Electromyography amplitude of three processed surface Electromyography signals measured as a function of time, according to certain exemplary embodiments; and,

FIG. 9 shows a graph of a sound amplitude of two processed sound signals measured as a function of time, according to certain exemplary embodiments.

### DETAILED DESCRIPTION

Disclosed herein is a system and method for collecting real-time data relating to a swallowing process of a subject, according to certain exemplary embodiments.

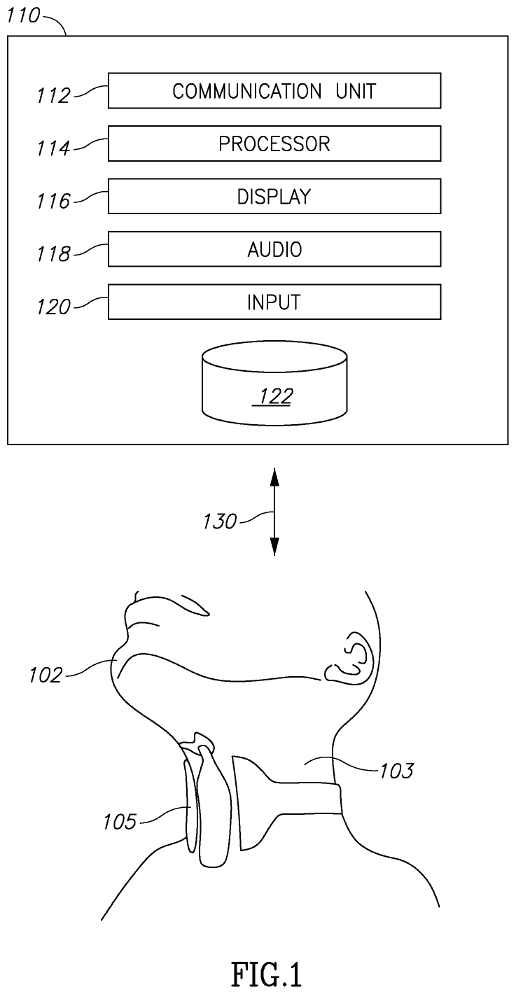

FIG. 1 shows a system 100 having a wearable device 105 for collecting real-time data relating to deglutition of a subject 102, according to certain embodiments. In some embodiments, wearable device 105 is configured to allow positioning of wearable device 105 on or adjacent to a throat 103 of subject 102. Wearable device 105 is connected to a computer device 110 to allow for real-time continuous communication between wearable device 105 and computer device 110. In some embodiments, computer device 110 can be a desktop, laptop, smartphone, tablet, server, or the like.

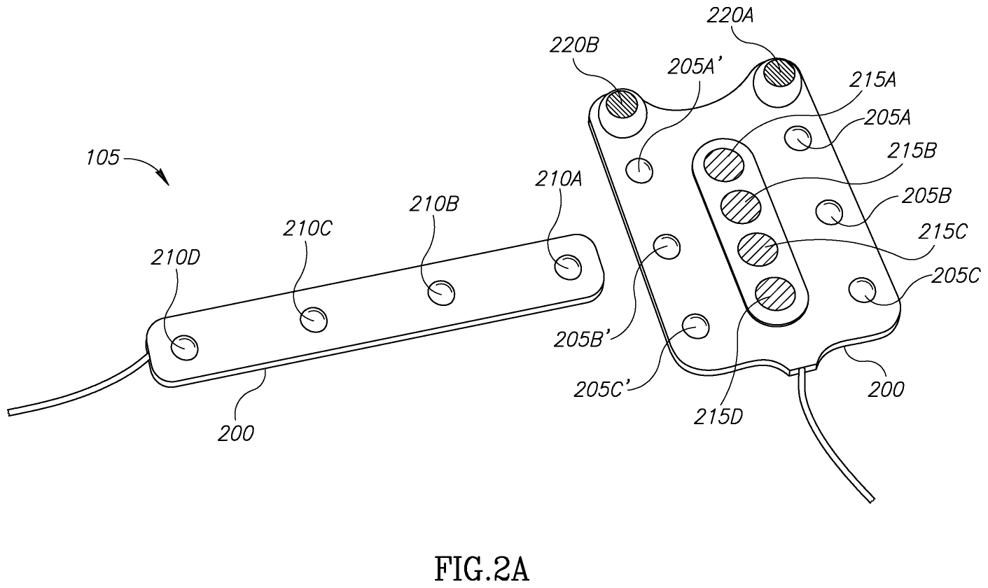

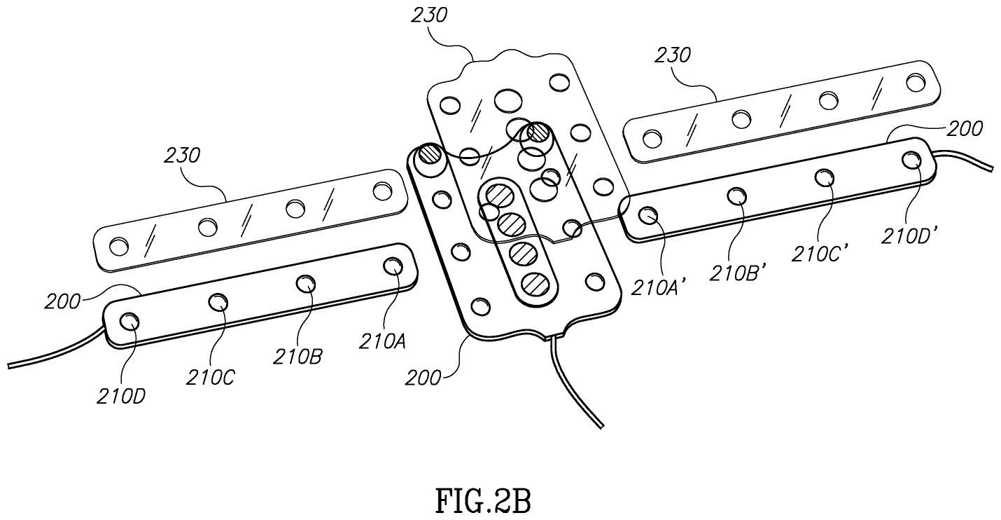

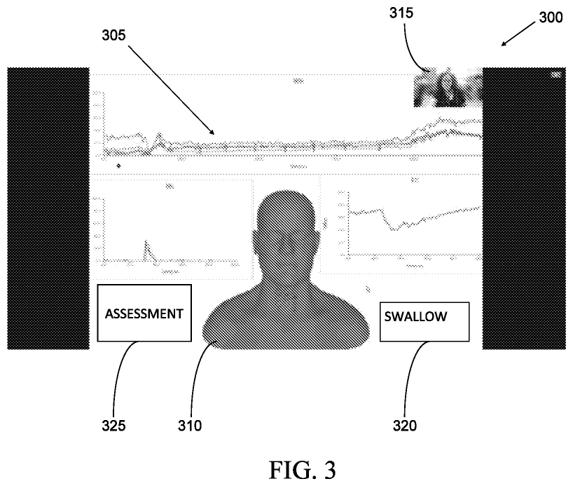

Computer device 110 includes a communication unit 112 configured to facilitate the continuous real-time communication between wearable device 105 and computer device 110, for example through wireless or wired connection therebetween, generally represented by arrows 130. Computer device 110 includes a processor 114 configured to operate, receive and assess the signals collected by sensors of wearable device 105 as further described in conjunction with FIG. 2A. In some embodiments, computer device 110 can include a display 116 to present a user interface 300 (FIG. 3) to subject 102 or to a third party (not shown), such as a therapist. In some embodiments, display 116 can show subject 102 a real-time signal collected by wearable device 105, present to subject 102 instructions and an assessment output about a dysphagia condition of subject 102, or the like.

In some embodiments, computer device 110 can include an audio unit 118 configured to provide audio feedback. For example, providing subject 102 with audio instructions to perform predetermined deglutition activities. In some embodiments, computer device 110 can include an input 120 configured to enable a third party, such as a therapist to input instructions for subject 102 or observations and data computer device 110 may require for generating an assessment output regarding a dysphagia condition of subject 102. For example, the user is a therapist providing instructions to the subject to perform predefined exercises. In some embodiments, input 120 can be a camera configured to capture real-time video or images of subject 102. In some embodiments, the camera may record a three-dimensional (“3D”) capture, for example, capturing a 3D image of subject 102 using two sensors or cameras. Computer device 110 includes a memory 122.

FIGS. 2A-2B schematically illustrate wearable device 105 of FIG. 1, according to certain exemplary embodiments. Referring to FIG. 2A, wearable device 105 includes a plurality of sensors for collecting signals to obtain real-time data relating to the swallowing performance of subject 102 (FIG. 1), according to certain exemplary embodiments. Wearable device 105 includes strapping 200 configured to position wearable device 105 around neck 103 (FIG. 1) near a larynx and chin of subject 102. In some embodiments, wearable device 105 includes one or more surface Electromyograph (“EMG”) sensors 205A, 205B, 205C, 205A′, 205B′, 205C′ configured to receive signals relating to electrical potential in tissue of the throat. In some embodiments, wearable device 105 includes one or more bio-impedance sensors 210A, 210B, 210C, 210D configured to receive signals relating to electric current flow in tissue of the throat in response to variable electric potentials. In some embodiments, wearable device 105 includes one or more bio-impedance sensors 210A, 210B, 210C, 210D configured to receive signals relating to variable electric potentials in response to electric current flow in tissue of the throat. In some embodiments, wearable device 105 includes one or more mechanical sensors 215A, 215B, 215C, 215D configured to receive signals relating to motion activity of the throat of the subject. In some embodiments, one or more mechanical sensors 215A, 215B, 215C, 215D can be accelerometers, strain sensors or the like. In some embodiments, wearable device 105 is configured to collect signals for calculating tomographic images of bio-impedance of the throat. In some embodiments, wearable device 105 includes one or more microphones 220A. 220B configured to record audio signals.

Referring to FIG. 2B, wearable device 105 can include an adhesive layer 230 for positioning and attaching wearable device 105 to the subject, according to certain exemplary embodiments. In some embodiments, adhesive layer 230 can be a double sided disposable medical adhesive layer to prevent contamination of wearable device 105 and to allow reuse by multiple subjects. In some embodiments, electro-conductive gel (not shown), adhesives, or the like, can be applied between sensors 205A, 205B, 205C, 205A′, 205B′, 205C′, 210A, 210B, 210C, 210D and the skin of subject 102.

FIG. 3 schematically illustrates a user interface 300, according to certain exemplary embodiments. In some embodiments, user interface 300 shows a real-time signal collected by wearable device 105 (FIG. 1), for example, EMG signal 305. In some embodiments, user interface 300 can present a video 315 or 3D capture 310 of subject 102. In some embodiments, user interface 300 can include instructions 320 that are presented to subject 102. For example, instructing subject 102 to swallow for a predefined duration. In some embodiments, user interface 300 can include an assessment output 325, for example, showing a numerical score evaluation of a dysphagia condition. In some embodiments, the user interface 300 can include a graphical illustration that represents the swallowing process, in order to provide feedback to the user. For example, the feedback is biofeedback, corresponding to the signals collected by wearable device 105 while subject is swallowing.

In some illustrative examples, user interface 300 can present a game or interactive activity to facilitate the rehabilitation subject 102. For example, the game can present different swallowing activities that subject 102 must complete while achieving a predetermined score. For example, The level of the game, or the graphical elements within the game correspond to predetermined measurements of the signals. In some embodiments, the measurements can include, for example, a time duration or amplitude of peaks or troughs of the EMG signal, the time delays between the peaks or troughs of the EMG signal collected from the same sensor or from different sensors, a correlation or a cross correlation between the EMG and bio-impedance signals, a metric including a combination of the collected signals, or the like.

In addition, an algorithm, based on machine-learning, is constructed based on the recorded signals, or features of the signals. For example, the algorithm can include features such as correlation, cross-correlation, differences, power spectra, Fast Fourier transform (“FFT”), or the like as the input for a machine-learning algorithm. The output of the algorithm is fed into the display and controls the features of the game.

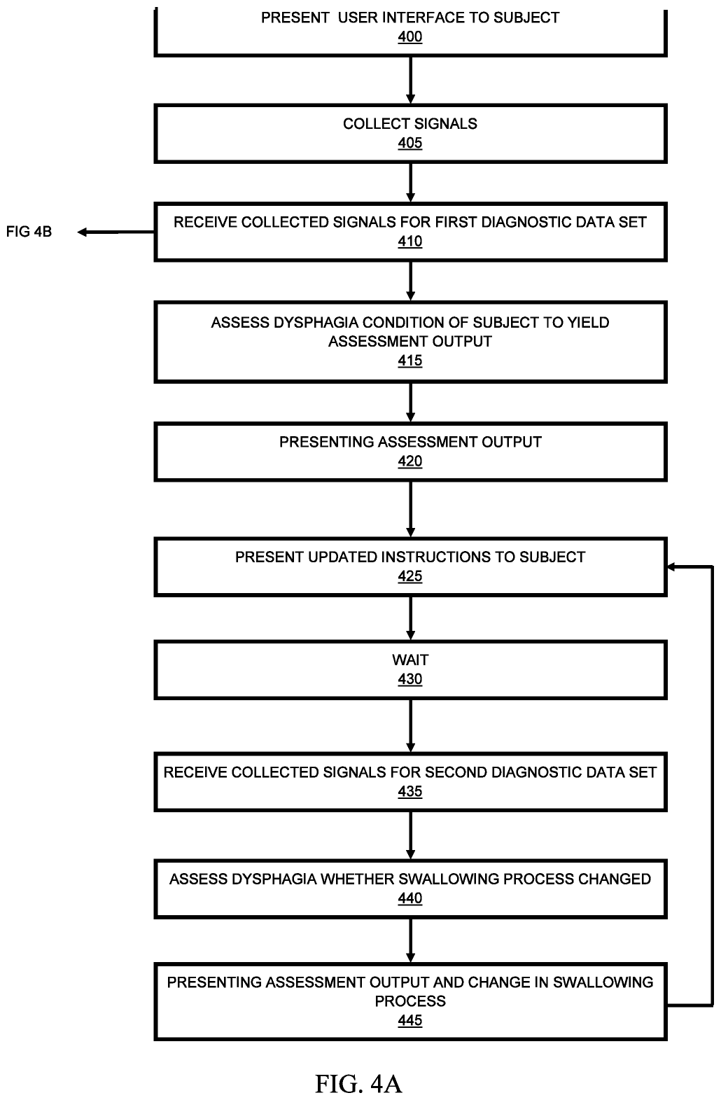

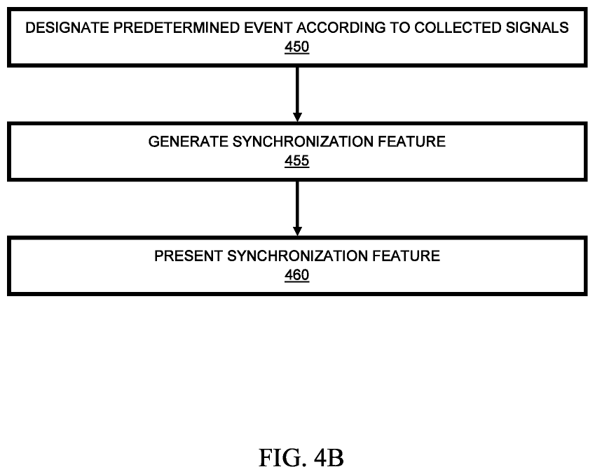

FIG. 4A outlines operations of a method for assessing dysphagia of subject 102 (FIG. 1), based on the measurement of the swallowing process, according to certain exemplary embodiments. In operation 400, processor 114 (FIG. 1) presents user interface 300 (FIG. 3) to subject 102. As described in conjunction with FIG. 3, user interface 300 can provide the instructions to subject 102 to swallow.

In operation 405, processor 114 operates wearable device 105 (FIG. 1) to collect signals. The sensors or wearable device collect a plurality of signals, such as EMG, bio-impedance, audio, or the like in real-time.

In operation 410, processor 114 receives the collected signals as a first diagnostic data set. The first data set includes the signals collected by wearable device 105.

In operation 415, processor 114 assess a swallowing process of the subject to yield an assessment output. Processor 114 analyzes the first data set to assess the swallowing process by determining how successful subject 102 was able to swallow according to the signals collected.

In operation 420, processor 114 presents assessment output, for example showing the assessment on display 116 (FIG. 1). Assessment output can be displayed in user interface 300 in an assessment display 325 (FIG. 3), for example, as a number value, as a graph, as a message or the like. In some embodiments, the assessment output can include a condition prediction, which shows a prediction of the improvement or the regression of the swallowing process.

In operation 425, processor 114 presents updated instructions to the subject 102. In some embodiments, the updated instructions are provided automatically by the software according to the assessment output to provide subject 102 with exercises or activities that will help improve the swallowing process. In some embodiments, the updated instructions can also be updated according to input provided by a third party, such as a therapist, via input 120 (FIG. 1). The input can include additional observations of the third party or additional activities for subject 102 to perform to improve the swallowing process.

In operation 430, processor 114 waits a predetermined time to allow the subject 102 to perform rehabilitation exercises and physical therapy. In some embodiments, processor 114 can wait a predetermined time to allow subject 102 to perform the activities that were provided in the instructions and to allow for sufficient repetitions of the activity to ensure a measurable change in the deglutition of subject 102.

In operation 435, processor 114 receives collected signals for a second diagnostic data set. The second diagnostic data set includes signals collected after the predetermined time thereby enabling processor to make a determination whether there was a change in the swallowing process of subject 102.

In operation 440, processor 114 assess the swallowing process to determine whether there was a change in the swallowing process.

In operation 445, processor 114 presents assessment output and change in swallowing process.

In some embodiments, processor 114 repeats operation 425 through operation 445 as many times as necessary during the session to collect sufficient data to determine the progress of the swallowing process, for example, whether there was improvement or deterioration of deglutition by subject 102.

Referring now to FIG. 4B, which outlines operations for synchronizing a predetermined event and the swallowing process, according to certain exemplary embodiments. In some embodiments, the predetermined event is a breathing cycle of subject 102 (FIG. 1). During the breathing cycle the volume of the sound recorded increases and decreases according to the passage of air through the larynx. During swallowing, the breathing cycle is interrupted. Therefore, in a subject that has a healthy swallow, the breathing cycle is automatically synchronized with the swallowing process. A subject with dysphagia may experience desynchronization of the breathing cycle and the swallowing process. In some embodiments, the predetermined synchronization event may be the elevation of the tongue, the closing of the vocal folds, the closing or opening of the upper esophagus sphincter the passage of a bolus of food through the upper esophagus or through specific fiduciary locations along the pharynx during the pharyngeal phase of swallowing. In some embodiments the predetermined event may be determined automatically by the processor, based on the collected signals or on features within the collected signals. In some embodiments the predetermined event is provided by the operator or by an external system (e.g. a metronome or a pace-maker).

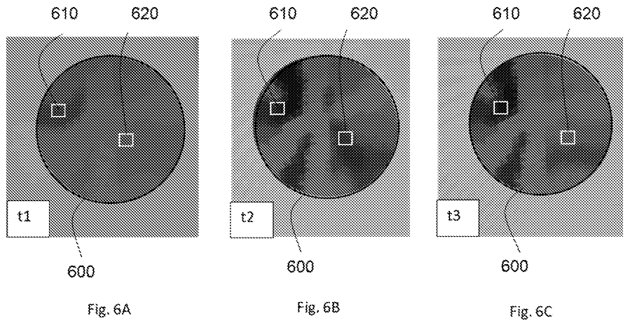

In operation 450, processor 114 (FIG. 1) designates a predetermined event according to the collected signals as described in conjunctions with FIGS. 6A-6C. 7, 8, 9. In operation 455, processor 114 generates a synchronization feature to provide an indication of when a swallowing process is going to occur as described in conjunction with FIG. 9.

In operation 460, processor 114 presents the synchronization feature via display 116 thereby to guide the user how to improve and/or change the synchronization thereby improving the swallowing sequence with regards to the predetermined event, such as the breathing cycle.

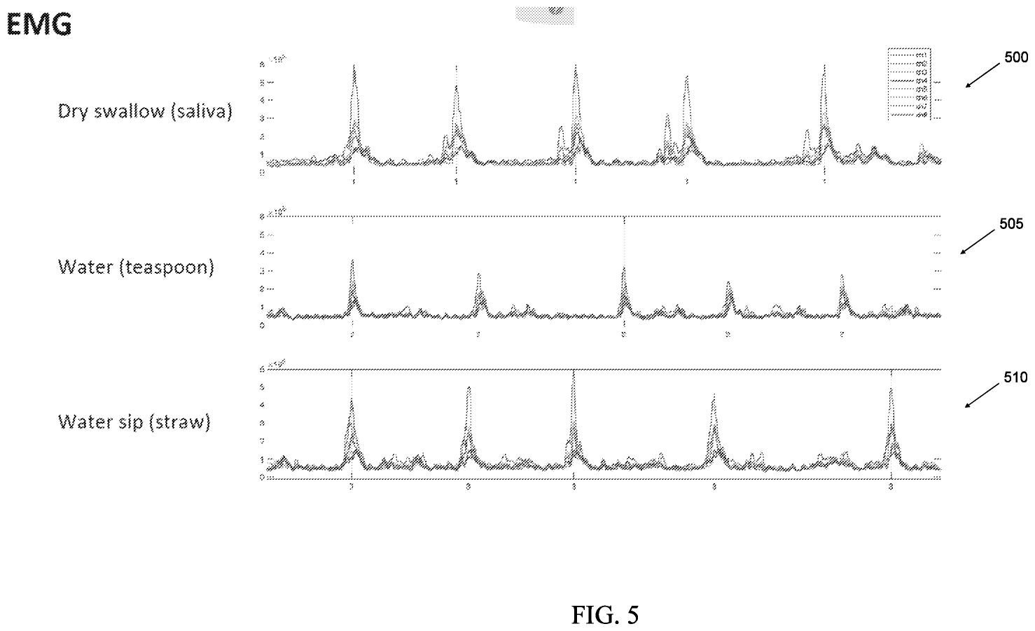

FIG. 5 shows three graphs of EMG signals collected for different deglutition activities, according to certain exemplary embodiments. A first graph 500 shows EMG signals for deglutition of saliva only. A second graph 505 shows EMG signals for deglutition of drinking a teaspoon of water. A third graph 510 shows EMG signals for deglutition of sipping water through a straw.

FIG. 6A-6C shows three-samples points on a bioimpedance tomography map 600, according to certain exemplary embodiments. In some embodiments, system 110 (FIG. 1) is configured to construct bioimpedance tomography (“bEIT”) map 600 according to data recorded by at least four electrodes 210A, 210B, 210C, 210D associated with the respective four electrode pairs 210A′, 210B′, 210C′, 210D′ (FIG. 2B). bEIT map 600 is calculated at each sample point, for example at a sampling rate of at least 10 Hertz (“Hz”). At each sample point, an amplitude or phase modulated current is applied between a pair of electrodes (e.g. any pair of 210A, 210B, 210C, 210D and 210A′, 210B′, 210C′, 210D′) positioned around the neck, and the voltage and/or potential difference is measured using different pair of electrodes positioned around the neck. The bioimpedance at each location within the sampling volume is calculated, for example according to the methods described in Seppänen, Aki, et al. “Electrical Impedance Tomography Imaging of Larynx.” Seventh International Workshop on Models and Analysis of Vocal Emissions for Biomedical Applications. 2011, incorporated herein by reference. A sequence of current “pairs” is applied at each sample point, by selecting a set of such current and voltage “pairs” that maps all possible combinations of selecting such pairs, or a subset of all possible combinations.

In some embodiments, several sample points of bEIT map 600 are generated from data recorded as a function of time from a plurality of electrodes, for example electrode pairs 210A. 210B, 210C, 210D and 210A′, 210B′, 210C′, 210D′ (FIG. 2A) positioned around the neck during swallowing. bEIT map 600 is calculated at different time points, represented by FIGS. 6A-6C, at three sample points t1,t2.13 shown as an example. A grey level of each pixel in bEIT map 600 is associated with an amplitude of the bioimpedance at each pixel. In some embodiments, for each bEIT map 600 the average amplitude at one or more regions of interests, referenced as 610, 620, are calculated for each sample point, or within a predetermined time window, for example, smaller than 0.1 seconds, which may include several sample points depending on the sample rate. In certain embodiments, region of interest 610 can be designated by a user or by system 110 according to predetermined parameters and modules executed by system 110.

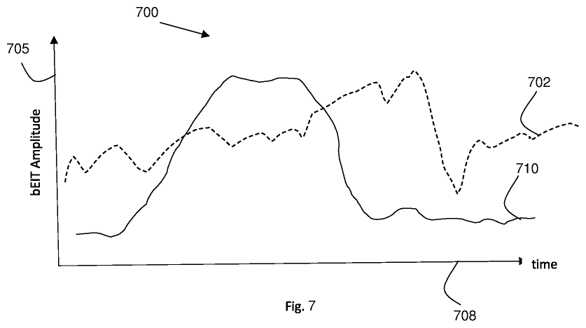

FIG. 7 shows a graph 700 showing an average amplitude 705 at exemplary regions of interest 610, 620 (FIGS. 6A-6C) as a function of time 708, according to certain exemplary embodiments. Signal 710 represents change in the amplitude of the bioimpedance within region of interest 610 and signal 702 represents changes in the bioimpedance within region of interest 620.

In certain embodiments, different regions of interest are designated with different sizes and shapes to facilitate calculating a predetermined feature, such as peak, median, average or the like, as a function of time. In certain embodiments, predetermined features of the region of bEIT map 600 are not specifically calculated within a predetermined region of interest, but are calculated based on features of bEIT map 600 that can be enhanced using image processing tools, such as contrast, standard deviation, kurtosis, or the like. In certain embodiments, the features of bEIT map 600 can be determined via machine-learning and deep learning methods. The determined features, such as signals 610, 620 are then analyzed to determine time dependent changes in the local amplitude of the bioimpedance within the throat during swallowing.

In certain embodiments, the features of bEIT map 600 as a function of time can enable defining phases of the swallowing process such as closing of the folds, passage of a bolus through the larynx, or the like. The different phases of the swallowing event exhibit changes in a predetermined feature as a function of time. This provides a time dependent signal that is related to changes in a local bioimpedance during swallowing.

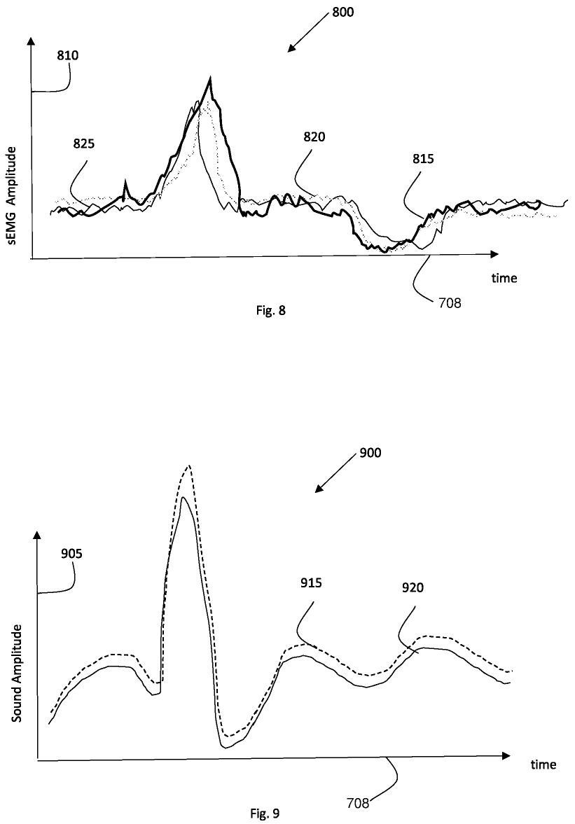

FIG. 8 shows a graph 800 of a surface electromyography amplitude 810 of three processed surface electromyography signals 815, 820, 825 measured as a function of time 708, according to certain exemplary embodiments. In some embodiments, analysis of signals 815, 820, 825 can includes rectification, band-pass filtering or the like. For example, signal 815 can be measured between electrode 205A and electrode 205A′ (FIG. 2A), signal 820 is measured between electrode 205B and electrode 205B′ (FIG. 2A), and signal 825 is measured between electrode 205C and electrode 205C′ (FIG. 2A). Features of signals 815, 820, 825, such as correlations between signals 815, 820, 825, time delay between local extreme of each signal or the like, are utilized to determine a metric for quantifying the signals 815, 820, 825 or a relation between the signals as a function of time.

FIG. 9 shows a graph 900 of a sound amplitude 905 of two processed sound signals 915, 920 measured as a function of time 708, according to certain exemplary embodiments. In some embodiments, two or more microphones 220A, 220B are positioned to record audio signals of a swallowing process. Sound signals 915, 920 are processed from the acquired analog voltage measured across the microphones, for example, by implementing low-pass filtering, band-pass filtering of the voltage signal, or the like. Microphones 220A, 220B are configured to record subtle sounds related to breathing and other sound sources associated with swallowing, such as the closing of the vocal folds. In certain embodiments, system 110 (FIG. 1) is configured to utilize sound signals 910, 915 to synchronize signals 702, 710 (FIG. 7), and signals 815, 820, 825 (FIG. 8) relative to a specific swallowing related signal, for example, closure of the vocal folds. In some embodiments, a feature of the differences of between signals 702, 710 or a cross-correlation between two signals, or the like, can be utilized as a synchronization feature. In some embodiments, a metric generated through a calculation of the signal modalities, such as bEIT, surface electromyography amplitude, sound, or the like, can be utilized as the synchronization feature.

In certain embodiments, a swallowing signal can be synchronized with the breathing cycle of the subject. For example, the breathing cycle-inhalation and exhalation can be determined according to sound signal 910, 915 (FIG. 9). A synchronization feature that depends on a time delay, a cross correlation or the like, can be determined from signals 702, 710, surface electromyography signals 815, 820, 825 and sound signals 910, 915, or from a signal calculated using one or more measurements of these signals, the cross correlation of the signals, a machine-learning based feature, or the like. The synchronization feature can be displayed to guide the user how to improve and/or change the synchronization thereby improving the swallowing sequence with regards to the breathing cycle. In some embodiments, a respiration sensor, such as a nasal sensor, a temperature sensor or the like, can be configured to record a signal associated with the breathing cycle.

In the context of some embodiments of the present disclosure, by way of example and without limiting, terms such as ‘operating’ or ‘executing’ imply also capabilities, such as ‘operable’ or ‘executable’, respectively.

Conjugated terms such as, by way of example, ‘a thing property’ implies a property of the thing, unless otherwise clearly evident from the context thereof.

The terms ‘processor’ or ‘computer’, or system thereof, are used herein as ordinary context of the art, such as a general purpose processor or a micro-processor, RISC processor, or DSP, possibly comprising additional elements such as memory or communication ports. Optionally or additionally, the terms ‘processor’ or ‘computer’ or derivatives thereof denote an apparatus that is capable of carrying out a provided or an incorporated program and/or is capable of controlling and/or accessing data storage apparatus and/or other apparatus such as input and output ports. The terms ‘processor’ or ‘computer’ denote also a plurality of processors or computers connected, and/or linked and/or otherwise communicating, possibly sharing one or more other resources such as a memory.

The terms ‘software’, ‘program’, ‘software procedure’ or ‘procedure’ or ‘software code’ or ‘code’ or ‘application’ may be used interchangeably according to the context thereof, and denote one or more instructions or directives or circuitry for performing a sequence of operations that generally represent an algorithm and/or other process or method. The program is stored in or on a medium such as RAM, ROM, or disk, or embedded in a circuitry accessible and executable by an apparatus such as a processor or other circuitry.

The processor and program may constitute the same apparatus, at least partially, such as an array of electronic gates, such as FPGA or ASIC, designed to perform a programmed sequence of operations, optionally comprising or linked with a processor or other circuitry.

The term computerized apparatus or a computerized system or a similar term denotes an apparatus comprising one or more processors operable or operating according to one or more programs.

As used herein, without limiting, a module represents a part of a system, such as a part of a program operating or interacting with one or more other parts on the same unit or on a different unit, or an electronic component or assembly for interacting with one or more other components.

As used herein, without limiting, a process represents a collection of operations for achieving a certain objective or an outcome.

As used herein, the term ‘server’ denotes a computerized apparatus providing data and/or operational service or services to one or more other apparatuses.

The term ‘configuring’ and/or ‘adapting’ for an objective, or a variation thereof, implies using at least a software and/or electronic circuit and/or auxiliary apparatus designed and/or implemented and/or operable or operative to achieve the objective.

A device storing and/or comprising a program and/or data constitutes an article of manufacture. Unless otherwise specified, the program and/or data are stored in or on a non-transitory medium.

In case electrical or electronic equipment is disclosed it is assumed that an appropriate power supply is used for the operation thereof.

The flowchart and block diagrams illustrate architecture, functionality or an operation of possible implementations of systems, methods and computer program products according to various embodiments of the present disclosed subject matter. In this regard, each block in the flowchart or block diagrams may represent a module, segment, or portion of program code, which comprises one or more executable instructions for implementing the specified logical function(s). It should also be noted that, in some alternative implementations, illustrated or described operations may occur in a different order or in combination or as concurrent operations instead of sequential operations to achieve the same or equivalent effect.

The corresponding structures, materials, acts, and equivalents of all means or step plus function elements in the claims below are intended to include any structure, material, or act for performing the function in combination with other claimed elements as specifically claimed. As used herein, the singular forms “a”, “an” and “the” are intended to include the plural forms as well, unless the context clearly indicates otherwise. It will be further understood that the terms “comprises” and/or “comprising” and/or “having” when used in this specification, specify the presence of stated features, integers, steps, operations, elements, and/or components, but do not preclude the presence or addition of one or more other features, integers, steps, operations, elements, components, and/or groups thereof.

As used herein the term “configuring” and/or ‘adapting’ for an objective, or a variation thereof, implies using materials and/or components in a manner designed for and/or implemented and/or operable or operative to achieve the objective.

Unless otherwise specified, the terms ‘about’ and/or ‘close’ with respect to a magnitude or a numerical value implies within an inclusive range of-10% to +10% of the respective magnitude or value.

Unless otherwise specified, the terms ‘about’ and/or ‘close’ with respect to a dimension or extent, such as length, implies within an inclusive range of-10% to +10% of the respective dimension or extent.

Unless otherwise specified, the terms ‘about’ or ‘close’ imply at or in a region of, or close to a location or a part of an object relative to other parts or regions of the object.

When a range of values is recited, it is merely for convenience or brevity and includes all the possible sub-ranges as well as individual numerical values within and about the boundary of that range. Any numeric value, unless otherwise specified, includes also practical close values enabling an embodiment or a method, and integral values do not exclude fractional values. A sub-range values and practical close values should be considered as specifically disclosed values.

As used herein, ellipsis ( . . . ) between two entities or values denotes an inclusive range of entities or values, respectively. For example, A . . . Z implies all the letters from A to Z, inclusively.

The terminology used herein should not be understood as limiting, unless otherwise specified, and is for the purpose of describing particular embodiments only and is not intended to be limiting of the disclosed subject matter. While certain embodiments of the disclosed subject matter have been illustrated and described, it will be clear that the disclosure is not limited to the embodiments described herein. Numerous modifications, changes, variations, substitutions and equivalents are not precluded.

Terms in the claims that follow should be interpreted, without limiting, as characterized or described in the specification.

1. A multi-modal sensor system, comprising,
a wearable device configured to receive signals relating to a swallowing process of a subject, the wearable device comprising:
at least one surface Electromyograph sensor configured to receive signals relating to electrical potential in muscles of the throat;
at least one bio-impedance sensor configured to receive signals relating to the bio-impedance of the tissues and structures within the throat;
at least one memory; and
at least one processor configured to:
operate at least one sensor of said wearable device;
synchronize the signals to at least one predetermined event to generate a synchronization feature;
receive the signals as a first diagnostic data set;
analyze said first diagnostic data set;
assess, based on the analysis, the swallowing process of the subject to yield an assessment output; and,
present said assessment output.

2. The multi-modal sensor system of claim 1, wherein said at least one bio-impedance sensor is configured to receive signals relating to electric current flow in tissue of the throat in response to application of variable electric potential and said at least one processor is further configured to designate the signals received from said at least one bio-impedance sensor as bio-impedance signals.

3. The multi-modal sensor system of claim 1, wherein said at least one bio-impedance sensor is configured to receive signals related to biopotential in response to current flow in tissue of the throat and said at least one processor is further configured to designate the signals received from said at least one bio-impedance sensor as a bio-impedance signals.

4. The multi-modal sensor system of claim 1, wherein said wearable device further comprises:
at least one mechanical sensor configured to receive signals relating to motion activity of the throat of the subject; and,
at least one microphone configured to collect audio signals relating to the throat of the subject.

5. A multi-modal sensor system of claim 1, wherein said at least one processor is further configured to analyze said bio-impedance signals to generate a time dependent tomographic map of the bio-impedance of a cross section of the throat.

6. The multi-modal sensor system of claim 1, wherein said assessment output includes a relation between the signals selected from the list which consists of: surface Electromyography, bio-impedance, mechanical and audio signals.

7. (canceled)

8. The multi-modal sensor system of claim 1, wherein said at least one processor is further configured to:
wait a predetermined time period;
receive collected signals for a second diagnostic data set;
assess, by analyzing said second diagnostic data set and comparing with said first diagnostic data set, whether the swallowing process changed; and,
generate a second assessment output indicating progress of the swallowing process.

9. The multi-modal sensor system according to claim 8, wherein said processor if further configured to:
initiate a user interface facilitate instructing the subject with a predetermined treatment; and,
updating instructions for the subject according to progress of the subject and said second assessment output.

10. The multi-modal sensor system of claim 1, wherein said processor is further configured to providing updated instructions according to said assessment output and input of a user.

11. (canceled)

12. (canceled)

13. The multi-modal sensor system of claim 1, further comprising a wireless communication unit configured to facilitate communication between said at least one processor and said at least one surface Electromyograph, at least one bio-impedance sensor, and at least one mechanical sensor and at least one audio sensor.

14. (canceled)

15. (canceled)

16. (canceled)

17. The multi-modal sensor system of claim 1, wherein said wearable device further comprises a double-sided disposable adhesive surface to facilitate fastening said wearable device to the neck throat of the subject.

18. The multi-modal sensor system of claim 1, wherein said at least one bio-impedance sensor comprises a plurality of bio-impedance sensors positioned to surround the throat at least 300 degrees.

19. The multi-modal sensor system of claim 1, wherein said at least one surface Electromyograph and said at least one mechanical sensor are positioned adjacent to a Larynx of the subject.

20. The multi-modal sensor system of claim 1, wherein analysis of the signal comprises measuring predetermined parameters of the signal.

21. The multi-modal sensor system according to claim 20, wherein said analysis further comprises determining a correlation between at least two signals of the signals collected.

22. (canceled)

23. (canceled)

24. (canceled)

25. (canceled)

26. A method comprising using at least one hardware processor for:
operate at least one sensor of said wearable device;
synchronizing the signals to at least one predetermined event to generate a synchronization feature;
receiving the signals as a first diagnostic data set;
analyzing said first diagnostic data set;
assessing, based on the analysis, the swallowing process of the subject to yield an assessment output; and,
presenting said assessment output.

27. The method according to claim 26, further comprising using the at least one processor for:
waiting a predetermined time period;
receiving collected signals for a second diagnostic data set;
assessing, by analyzing said second diagnostic data set and comparing with said a first diagnostic data set, whether the swallowing process changed; and,
generating a second assessment output indicating progress of the subject.

28. The method according to claim 27, further comprising using the at least one processor for:
initiating a user interface to facilitate instructing the subject with a predetermined treatment; and,
updating instructions for the subject according to progress of the subject and said second assessment output.

29. The method according to claim 28, wherein said signals are collected by a wearable device comprising:
at least one surface Electromyograph configured to receive signals relating to electrical potential in tissue of the throat; and,
at least one bio-impedance sensor configured to receive signals relating to electric current flow in response to application of variable electric potential in tissue of the throat;

30. The method according to claim 29, wherein said wearable device further comprises:
at least one mechanical sensor configured to receive signals relating to motion activity of the throat of the subject; and,
at least one microphone configured to collect audio signals relating to the throat of the subject.

31. (canceled)

32. (canceled)

33. (canceled)

34. (canceled)
